(19) United States
(12) Patent Application Publication
(10) Pub. No.: US 2019/0133896 A1
(43) Pub. Date: May 9, 2019

(54) EMULSIFIED COMPOSITION AND COSMETIC USING SAME

(71) Applicant: KANEKA CORPORATION, Osaka (JP)

(72) Inventors: Tadao Tsuji, Osaka (JP); Toshiyuki Masuda, Osaka (JP); Kisaburo Noguchi, Osaka (JP)

(73) Assignee: KANEKA CORPORATION, Osaka (JP)

(21) Appl. No.: 16/233,781

(22) Filed: Dec. 27, 2018

Related U.S. Application Data
(63) Continuation of application No. PCT/JP2017/024552, filed on Jul. 4, 2017.

(30) Foreign Application Priority Data
Jul. 5, 2016 (JP) 2016-133023

(51) Int. Cl. A61K 8/06; A61K 8/898; A61K 8/44

(52) U.S. Cl. CPC A61K 8/06; A61K 2800/10; A61K 8/44; A61K 8/898

(57) ABSTRACT

An emulsified composition includes an amino-modified silicone oil, an aqueous solvent, and a surfactin. The surfactin is represented by the following formula:

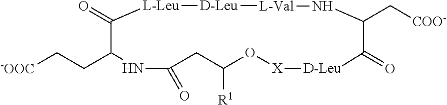

where X is an amino acid residue selected from the group consisting of a leucine residue, an isoleucine residue, and a valine residue and R¹ is a C₉₋₁₈ alkyl group.

### TECHNICAL FIELD

One or more embodiments of the present invention relate to an emulsified composition having excellent emulsifying characteristics and a cosmetic containing the emulsified composition.

### BACKGROUND

Silicone has a structure in which mostly the side chains or polysiloxane are substituted with lower alkyl groups. By having excellent heat resistance or the like and also high safety to human body, and for exhibiting various property states such as an oil state, a rubber state, and resin state depending on molecular weight or the like, silicone is widely used in many fields. For example, as silicone oil exhibits an excellent lubricating property or water repellency, for the purpose of providing skin with fresh use feel by suppressing stickiness or for the purpose of providing hair with silky texture by enhancing the softness, silicone oil is blended with other oily components in a cosmetic.

However, silicone is chemically inactive and has low affinity for other compounds in some cases. As such, amino-modified silicone having an amino substituent introduced to a side chain is being developed. From the viewpoint that the amino-modified silicone is chemically active, the amino-modified silicone is used as an adhesive component or even for a coatable silicone sealant. Furthermore, from the viewpoint that the amino-modified silicone has an improved adhesion property and can provide flexibility or a lubricating property, the amino-modified silicone is also used as a component of a hair cosmetic for improving smoothness or preventing entanglement. Other than those, in view of the adsorption property or reactivity, the amino-modified silicone is also used for wax or the like.

Furthermore, silicone oil may have low compatibility with oily components, rendering it difficult to form an emulsified system. As such, for a composition containing silicone oil, it is generally necessary to blend a surfactant in relatively large amount.

In Patent Document 1 and Patent Document 2, for example, a hair cosmetic containing biosurfactant and silicones is described, and, as one of specific examples of silicone, amino-modified silicone is used. Furthermore, in Patent Document 3, a thickened gel-like oily composition containing an anionic surfactant having a lipopeptide structure and an oily component is disclosed, and, as a specific example of the anion surfactant, a surfactin, which is a natural biosurfactant, is used.

### Patent Document 1: Japanese Unexamined Patent Application, Publication No. 2011-26280

### Patent Document 2: Japanese Unexamined Patent Application, Publication No. 2011-26281

Patent. Document 3: Japanese Unexamined Patent Application, Publication No. 2003-176211

As described above, a surfactant is generally used in a composition containing silicone oil.

However, there is a case in which such a composition cannot stably maintain the emulsified state even if the composition is in an emulsified state immediately after the production. As such, to maintain the emulsified state of a composition containing silicone oil, a method of increasing the blending amount of a surfactant, reducing the amount of silicone oil or oily components, or increasing the viscosity of an aqueous phase has been conventionally employed. However, this method may yield an increased irritating property or limit the constituents of a composition.

In this regard, one or more embodiments of the present invention provide an emulsified composition that is stably maintained in an emulsified state, and a cosmetic containing the emulsified composition.

### SUMMARY

The present inventors have repeated intensive studies. As a result, the present inventors have found that, for stably maintaining the emulsified state of a composition containing an amino-modified silicone oil, which is useful as a component of a hair cosmetic or the like, surfactin as one kind of biosurfactants is quite suitable, and completed one or more embodiments of the present invention accordingly.

Hereinbelow, one or more embodiments of the present invention are described.

[1] An emulsified composition containing an amino-modified silicone oil, an aqueous solvent, and a surfactin represented by the formula (I) below:

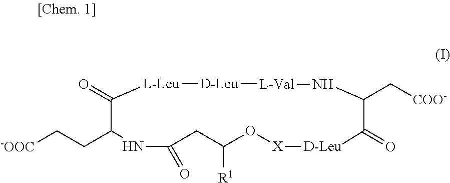

[in the formula,

X represents an amino acid residue selected from the group consisting of a leucine residue, an isoleucine residue, and a valine residue; and

R¹ represents a C₉₋₁₈ alkyl group].

[2] The emulsified composition described in above [1], further containing an oily component.

[3] The emulsified composition described in above [1] or [2], in which a concentration of the surfactin is 0.1 mM or more and 50 mM or less with respect to the entire emulsified composition.

[4] A cosmetic containing the emulsified composition described in any one of above [1] to [3].

In the emulsified composition according to one or more embodiments of the present invention, the emulsified state is stably maintained at even less amount of a surfactant while the composition contains an amino-modified silicone oil. Furthermore, from the viewpoint that the surfactin as a surfactant to be blended is the emulsified composition according to one or more embodiments of the present invention is a natural peptide surfactant, the surfactin has low skin permeability, and thus considered to have low transdermal toxicity compared to synthetic surfactants that are universally used. Furthermore, even when released to an environment, the surfactin is rapidly degraded by microorganisms or the like, and thus has a low load given to an environment. Accordingly, the emulsified composition according to one or more embodiments of the present invention has safety and high product value, and thus can be suitably used for a cosmetic or the like.

### BRIEF DESCRIPTION OF THE DRAWINGS

FIG. 1 is a graph showing the result of an experiment for testing the emulsified state-maintaining action of sodium surfactin on a composition containing water and various silicone oils.

FIG. 2 a graph showing the result of an experiment for testing the emulsified state-maintaining action of sodium dodecyl sulfate on a composition containing water and various silicone oils.

FIG. 3 is a graph showing the result of an experiment for testing the emulsified state-maintaining action of low-concentration sodium surfactin on a composition containing water and various silicone oils.

FIG. 4 is a graph showing the result of an experiment for testing the emulsified state-maintaining action of low-concentration sodium dodecyl sulfate on a composition containing water and various silicone oils.

FIG. 5 is a graph showing the result of an experiment for testing the emulsified state-maintaining action of sodium surfactin or sodium decyl sulfate on a composition containing water and an amino-modified silicone oil.

FIG. 6 is a graph showing the result of an experiment for testing the emulsified state-maintaining action of low-concentration sodium surfactin or sodium decyl sulfate on a composition containing water and an amino-modified silicone oil.

### DETAILED DESCRIPTION OF THE EMBODIMENTS

An emulsified composition according to one or more embodiments of the present invention is characterized by containing an amino-modified silicone oil, an aqueous solvent, and a surfactin represented by the formula (I) above.

In general, the amino-modified silicone oil of one or more embodiments is a compound which is liquid at room temperature, in which part of the alkyl group in C₁₋₄ alkylpolysiloxane is substituted with an amino substituent. Examples of the amino substituent of the amino-modified silicone oil may include, but are not limited to, an amino group (−NH₂) a mono (C₁₋₄ alkyl)amino group, a di (C₁₋₄ alkyl) amino group, an amino-C₁₋₄ alkylene group, a mono (C₁₋₄ alkyl) amino-C₁₋₄ alkylene group, a di (C₁₋₄ alkyl) amino-C₁₋₄ alkylene group, an amino-C₁₋₄ alkylene-amino-C₁₋₄ alkylene group, a mono (C₁₋₄ alkyl) amino-C₁₋₄ alkylene-amino-C₁₋₄ alkylene group, and a di (C₁₋₄ alkyl) amino-C₁₋₄ alkylene-amino-C₁₋₄ alkylene group. Furthermore, as long as the substitution is given at the amino substituent, it can be substituted with other substituents. Examples of other substituents may include, but are not limited to, a C₁₋₄ alkyl-C₂₋₄ alkylene glycol residue-C₁₋₄ alkylene group. In a di (C₁₋₄ alkyl)amino group, an amino-C₁₋₄ alkylene-amino-C₁₋₄ alkylene group, or the like, plural C₁₋₄ alkyl groups may be the same as or different from each other, and plural C₁₋₄ alkylene groups may be the same as or different from each other. The same holds true for a C₂₋₄ alkylene glycol residue, and it may be either a polyethylene glycol residue or a polypropylene glycol residue, and it may be also an ethylene glycol-propylene glycol copolymer residue. Incidentally, in the present specification, the “glycol residue” indicates an atomic group that remains after excluding hydrogen atoms from two alcohol hydroxyl groups that are included in glycol, and the “glycol copolymer residue” indicates an atomic group that remains after excluding hydrogen atoms from two alcohol hydroxyl groups that are included in the glycol copolymer. Only one kind of the amino-modified silicone oil may be used, or two or more kinds of the amino-modified silicone oils may be used.

In one or more embodiments of the present invention, examples of the “C₁₋₄ alkyl group” may include a methyl group, an ethyl group, an n-propyl group, an isopropyl group, and an n-butyl group, a methyl group or an isopropyl group is preferable, and a methyl group is more preferable. Examples of the “C₁₋₄ alkylene group” may include a methylene group, an ethylene group, an n-propylene group, and an n-butylene group, and a C₁₋₂ alkylene group is preferable.

In one or more embodiments, the molecular weight or polymerization degree of the amino-modified silicone oil may be suitably selected depending on desired viscosity or the like of the amino-modified silicone oil itself or emulsified composition. For example, it is possible to select an amino-modified silicone oil of which viscosity at 25° C. is about 500 mm²/s or more and 10000 mm²/s or less. From the viewpoint of the obtainability of the amino-modified silicone oil, handling property of an emulsified composition to be obtained, or the like, the viscosity is preferably 500 mm²/s or more and 2000 mm²/s or less, more preferably 600 mm²/s or more and 1500 mm²/s or less, and even more preferably 700 mm²/s or more and 1000 mm²/s or less.

Blending amount of the amino-modified silicone oil is not particularly limited, and may be suitably adjusted. However, for example, the blending amount of one or more embodiments can be set to 0.1% by mass or more and 80% by mass or less with respect to the entire emulsified composition. As the ratio is 0.1% by mass or more, characteristics of the amino-silicone oil can be more certainly exhibited. in the emulsified composition. Meanwhile, the ratio is preferably 80% by mass or less, since easy emulsification can be readily obtained if the ratio is not excessively high. The ratio is more preferably 0.5% by mass or more, and even more preferably 1.0% by mass or more. Furthermore, the ratio is more preferably 60% by mass or less or 50% by mass or less, and even more preferably 30% by mass or less or 20% by mass or less.

In one or more embodiments, the aqueous solvent is not particularly limited as long as it is a solvent which has water as a main component, and examples thereof may include water and a mixed solvent of water and a water-miscible organic solvent. Ratio of water in the mixed solvent may be suitably adjusted, but for example, the ratio can be set to 60% by volume or more or 70% by volume or more, and is more preferably 80% by volume or more or 90% by volume or more, and even more preferably 95% by volume or more. Upper limit of the ratio of water in the mixed solvent is not particularly limited, and the upper limit may be less than 100% by volume, 99.5% by volume, or 99% by volume. Incidentally, In the present specification, the ratio expressed with “% by volume” indicates a value at 20° C.

The “water-miscible organic solvent” indicates an organic solvent which can be homogeneously mixed in an amount of 5 g or more in 100 mL of water at 20° C., for example. Examples of the water-miscible organic solvent may include, but are not limited to, an alcohol-based solvent such as methanol, ethanol, or isopropanol; an ether-based solvent such as diethyl ether or tetrahydrofuran; a ketone-based solvent such as acetone; a nitrile-based solvent such as acetonitrile; an amide-based solvent such as dimethyl formamide or dimethyl acetamide; a sulfoxide-based solvent such as dimethyl sulfoxide; and a carboxylic acid-based solvent such as formic acid and acetic acid. Suitably, a water-miscible organic solvent that is miscible with water at any ratio is used.

In one or more embodiments, the blending amount of the aqueous solvent is not particularly limited, and may be suitably adjusted. However, the blending amount can be set to 50% by mass or more and 99% by mass or less with respect to the entire emulsified composition. As the ratio is 50% by mass or more, water-soluble components including surfactin (I) can be suitably dissolved. Meanwhile, the ratio is preferably 99% by mass or less, since easy emulsification can be readily obtained if the ratio is not excessively high. The ratio is more preferably 70% by mass or more, and even more preferably 80% by mass or more. Furthermore, the ratio is more preferably 95% by mass or less, and even more preferably 90% by mass or less.

The emulsified composition according to one or more embodiments of the present invention contains a surfactin that is represented by the following formula (I) (hereinafter abbreviated as “surfactin (I)”). In the emulsified composition according to one or more embodiments of the present invention, a dispersion state of the amino-modified silicone oil is significantly improved by blending of the surfactin (I), in particular, and thus it becomes possible to maintain stably the emulsified state. Furthermore, since the surfactin (I) is a peptide compound, the surfactin (I) has a small load applied to a natural environment, and thus is safe to a human body.

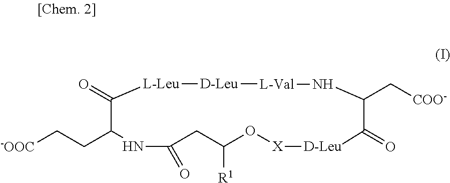

[in the formula,

X represents an amino acid residue selected from the group consisting of a leucine residue, an isoleucine residue, and a valine residue; and

R¹ represents a C₉₋₁₈ alkyl group]

The amino acid residue as X may be either L form or D form, but is preferably L form.

The “C⁹⁻¹⁸ alkyl group” indicates a linear or branched monovalent saturated hydrocarbon group with carbon atom number of 9 or more and 18 or less. Examples thereof include, but are not limited to, an n-nonyl group, a 6-methyloctyl group, a 7-methyloctyl group, an n-decyl group, a 8-methylnonyl group, an n-undecyl group, a 9-methyldecyl group, as n-dodecyl group, a 10-methylundecyl group, an n-tridecyl group, a 11-methyldodecyl group, an n-tetradecyl group, an n-pentadecyl group, an n-hexadecyl group, an n-heptadecyl group, and an n-octadecyl group.

In one or more embodiments, only one kind of the surfactin (I) may be used, or two or more kinds of the surfactin (I) may be used. For example, plural surfactins (I) which have a different C₉₋₁₈ alkyl group represented by R¹ may be used.

The surfactin (I) of one or more embodiments can be isolated from a culture solution after culturing bacterial strain belonging to Bacillus subtilis according to a known method, for example, and the surfactin (I) may be an adjusted product or a purified product or may be used as a non-purified product, for example, as a culture solution itself. Furthermore, a surfactin obtained by a chemical synthesis method can be also used in the same manner.

As a raw material of the surfactin (I), a salt of the surfactin (I) may be used in one or more embodiments. The counter cation M⁺, which constitutes a salt of the surfactin (I), is not particularly limited, but examples thereof include an alkali metal ion and a quaternary ammonium ion.

In one or more embodiments, the alkali metal ion which can be used for a salt of the surfactin (I) is not particularly limited, but examples thereof include a lithium ion, a sodium ion, and a potassium ion. Furthermore, in a case in which two or more kinds of alkali metal ions are used, they may he the same as or different from each other.

Examples of a substituent of the quaternary ammonium ion include, but are not limited to, an organic group like a C₁₋₄ alkyl group such as a methyl group, an ethyl group, an n-propyl group, an isopropyl group, an n-butyl group, or a t-butyl group; a C₇₋₁₄ aralkyl group such as a benzyl group, a methylbenzyl group, or a phenylethyl group; and a C₆₋₁₂ aryl group such as a phenyl group, a toluyl group, or a xylyl group. Examples of the quaternary ammonium ion include, but are not limited to, a tetramethyl ammonium ion, a tetraethyl ammonium ion, and a pyridinium ion.

Incidentally, in a salt of the surfactin (I), the two counter ions may be the same as or different from each other. Furthermore, between two —COO⁻ that are present in the surfactin (I), it is also possible that one is in a state of —COOH or —COO⁻ and the tether is in a state of —COO⁻M⁺.

The blending amount of the surfactin (I) of one or more embodiments is not particularly limited, and may be suitably adjusted. However, the concentration of the surfactin (I) can be set to 0.1 mM or more and 50 mM or less with respect to the entire emulsified composition, for example. As the concentration is 0.1 mM or more, the emulsifying action by the surfactin (I) can be more certainly exhibited. Meanwhile, the concentration is probably 50 mM or less, since the effect by the surfactin (I) is hardly saturated if the ratio is not excessively high. The concentration is more preferably 0.5 mM or more, and even more preferably 1 mM or more. Furthermore, the concentration is more preferably 40 mM or less, and even more preferably 30 mM or less.

The emulsified composition according to one or more embodiments of the present invention may additionally contain an oily component. Since there is a case in which the emulsified composition according to one or more embodiments of the present invention has a more improved dispersion state of the amino-modified silicone oil according to blending of an oily component, maintaining stably the emulsified state is more easily achieved. The oily component used in one or more embodiments of the present invention is not particularly limited as long as it is not mixed with water at any ratio, and preferred examples thereof include hydrocarbons such as squalane, fluid paraffin, light fluid isoparaffin, ceresin, polyethylene powder, squalene, microcrystalline wax, Vaseline, fluid isoparaffin, polybutene, or mineral oil; waxes such as bee wax, carnauba wax, candelilla wax, jojoba oil, lanolin, or whale wax; oils and fats such as macadamia oil, olive oil, cotton seed oil, soy bean oil, avocado oil, rice bran oil, rice oil, rice germ oil, palm seed oil, castor oil, rosehip oil, evening primrose oil, camellia oil, horse oil, grape seed oil, palm oil, meadow foam oil, shear butter, corn oil, safflower oil, or sesame oil; esters such as ethylhexyl palmitate, isononyl isononanoate, isopropyl myristate, ethyl oleate, glyceryl tri(caprylate·capreate), cetyl 2-ethylhexanoate, glyceryl tri 2-ethylhexanoate, diisopropyl sebacate, or cholesteryl hydroxystearate; fatty acids such as myristic acid, stearic acid, or oleic acid; silicone oils such as methylpolysiloxane, methylphenylpolysiloxane, or amino-modified silicone; higher alcohols such as cetanol or oleyl alcohol; and alkyl glyceryl ethers such as batyl alcohol and chimyl alcohol. Only one kind of the oily component may be used, or two or more kinds of the oily component may be used.

In one or more embodiments of the present invention, the concentration of the oily component is preferably 0.5% by weight to 99% by weight, more preferably 0.88% by weight to 99% by weight, even more preferably 1% by weight to 98.88% by weight, and particularly preferably 10% by weight to 50% by weight. As the oily component is within the above concentration range, it becomes possible to form a stable emulsion.

The emulsified composition according to one or more embodiments of the present invention may be suitably blended with other addition components depending on the purpose of using the emulsified composition, or the like. Other addition components are not particularly limited, but examples thereof may include, but are not limited to, thickening polysaccharides such as guar gum and xanthan gum; celluloses such as hydroxypropyl cellulose and carboxymethyl cellulose; carboxyvinyl polymers such as an acrylic acid polymer or an acrylic acid copolymer; silicone compounds; coloring agents; pH adjusting agents; plant extracts; preservatives; chelating agents; vitamin preparations; pharmaceutical components such as an anti-inflammatory agent; fragrance; UV absorbing agents; and anti-oxidants. Furthermore, even in the emulsified composition according to one or more embodiments of the present invention or a product thereof, a conventional surfactant may be used in combination, in addition to the surfactin (I). However, it is preferable that all surfactants in the emulsified composition or product are the surfactin (I).

The product containing the emulsified composition according to one or more embodiments of the present invention is not particularly limited, and examples thereof may include those containing an amino-modified silicone oil like a cosmetic such as a cosmetic product for decoration, cream, gel, lotion, deodorant, or a UV blocking agent; a cosmetic product or a toiletry product such as shampoo, a shower bath product, an antiperspirant, a liquid toothpaste, or an oral cleanser; moisturized wipe such as a wet tissue which is used for cleansing cosmetics or wiping baby bottom; a sterilizing solution for sterilizing hands or the like for medical or household use; a fiber product; a rubber·plastic related product; a product of civil engineering·construction; a paper·pulp product; a mechanical·metal product; a cleaning product; a beverage or food product; a paint·ink product; a product for environment conservation; an agriculture·fertilizer product; a product for information industry; and other cleansing agents for industrial use.

A method for producing the emulsified composition according to one or more embodiments of the present invention is not particularly limited, and it is sufficient to have just mixing each component described above. However, to form an emulsified state, vigorous stirring by using a mixer, ultrasonic waves, or the like is preferable. The temperature at that time is sufficiently room temperature, and, specifically, may be set to about 10° C. or higher and 50° C. lower. The stirring time is not particularly limited, but may be set to about 10 seconds or longer and 1 hour or shorter, for example.

Since the emulsified composition according to one or more embodiments of the present invention has a stably maintained emulsified state mainly by the action of the surfactin (I), the emulsified composition is highly suitable for an application to a product containing an amino-modified silicone oil.

### EXAMPLES

Hereinbelow, one or more embodiments of the present invention will be described in more detail based on examples, but the present invention is not limited to those examples.

### Example 1

To a test tube, 2 g of silicone oil shown in Table 1 was added, 2 g of an aqueous solution of a surfactant, specifically, an aqueous solution of sodium surfactin or an aqueous solution of sodium dodecyl sulfate, was further added, and by mixing for 1 minute using a test tube mixer at room temperature, a mixture was obtained. Incidentally, the concentration of the aqueous solution of a surfactant was adjusted such that the final concentration is 25 mM. Furthermore, all of the silicone oils that have been used are products manufactured by Shin-Etsu Silicones.

TABLE 1

| | | Viscosity |
|---|---|---|
| Component name | Product name | (mm²/s) |
| Methylpolysiloxane | KF-96A-5cs | 3 |
| Methylpolysiloxane | KF-96A-20cs | 20 |
| Methylpolysiloxane | KF-96A-200cs | 200 |
| Methylpolysiloxane | KF-96A-500cs | 500 |
| Methylpolysiloxane | KF-96A-1000cs | 1000 |
| Methylpolysiloxane | KF-96A-3000cs | 3000 |
| Methylpolysiloxane | KF-96A-5000cs | 5000 |
| Highly-polymerized methylpolysiloxane | KF-96H-10000cs | 10000 |
| Decamethylcyclopentasiloxane | KF-995 | 4 |
| Aminoethylaminopropylmethylsiloxane | KF-8004 | 800 |
| dimethylsiloxane copolymer | | |

The mixture was left to stand still at 50° C., and the next day (i.e., 1 day later), 1 week later, 2 weeks later, and 3 weeks later, the whole height (total height of emulsified phase and aqueous phase) and the height of emulsified phase only were measured by using a height gauge. Then, an emulsification index was calculated based on the following formula.

Emulsification index (%)=[(Height of emulsified phase)/(Whole height)]×100

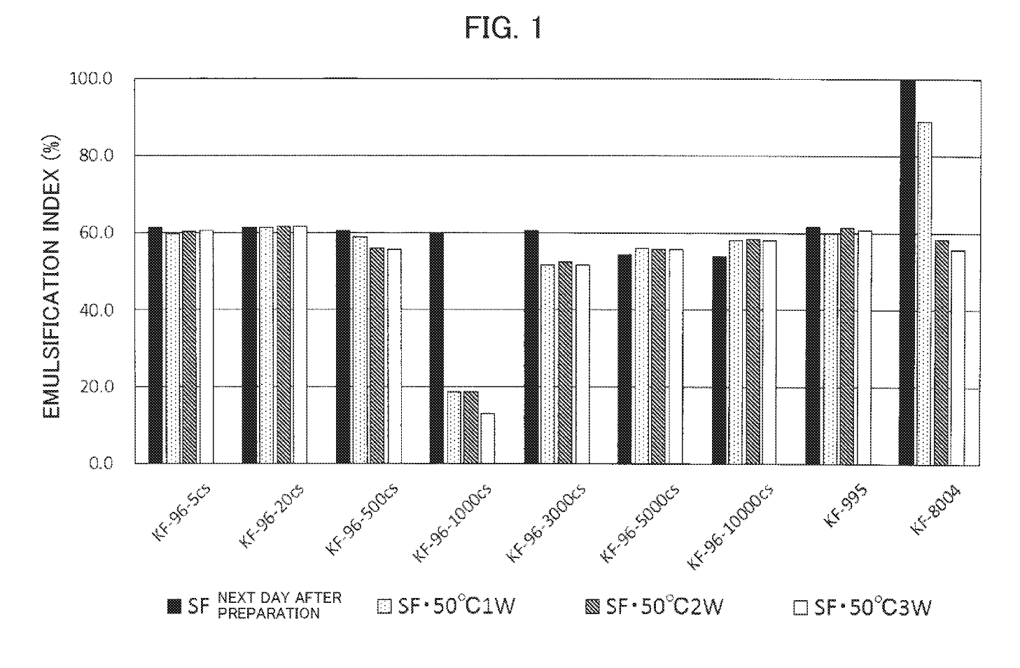

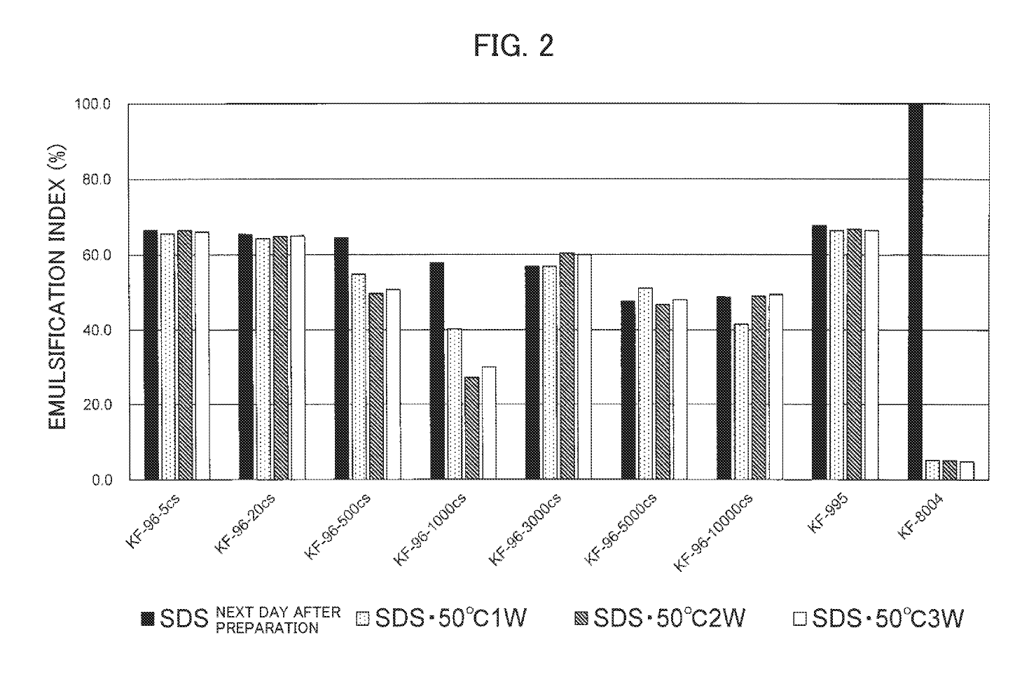

The result of using sodium surfactin is shown in FIG. 1 and the result of using sodium dodecyl sulfate is shown in FIG. 2. Incidentally, in FIG. 1, “SF” represents sodium surfactin, and, in FIG. 2, “SDS” represents sodium dodecyl sulfate.

Like the results shown in FIGS. 1 and 2, the emulsifying effect of sodium surfactin and sodium dodecyl sulfate hardly changes.

However, regarding the amino-modified silicone oil (product name of “KF-8004”), even if the emulsification can be initially achieved with sodium dodecyl sulfate, liquid separation is immediately yielded according to the still standing; on the other hand, it was evident that the emulsified state is relatively stably maintained when sodium surfactin is used. Accordingly, it was recognized that sodium surfactin tends to be suitable for emulsification by an amino-modified silicone oil.

### Example 2

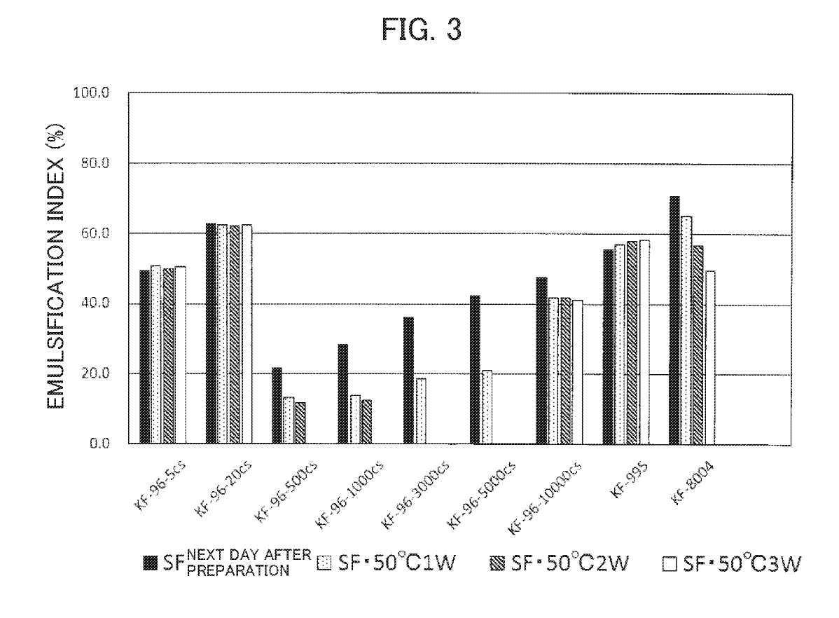

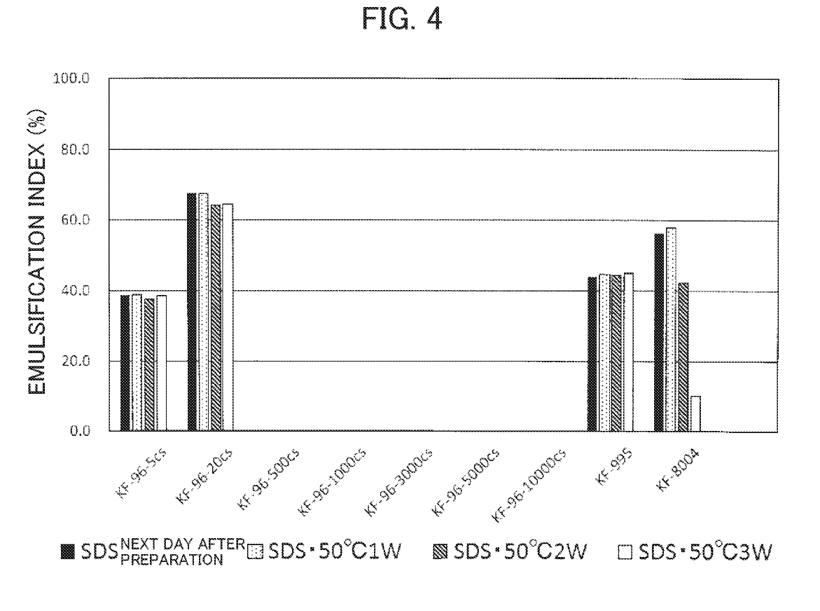

An experiment was carried out in the same manner as Example 1 above except that the final concentration of each surfactant was changed to 1/50, i.e., 0.5 mM. The result of using sodium surfactin is shown in FIG. 3 and the result of using sodium dodecyl sulfate is shown in FIG. 4.

Like the results shown in FIGS. 3 and 4, there is a tendency of generally having lower emulsifying effect in both of sodium surfactin and sodium dodecyl sulfate, and, in the case of sodium dodecyl sulfate, the emulsifying effect was not recognized at all depending on the type of the silicone oil.

In the case of the amino-modified silicone oil (product name of “KF-8004”), although the emulsifying effect was initially recognized even with sodium dodecyl sulfate, the emulsified state was not stable and separation was yielded over time.

On the other hand, in the case of blending sodium surfactin, a tendency of losing gradually the emulsified state was recognized, but the degree thereof was clearly lower compared to sodium dodecyl sulfate. As described above, even when the concentration of a surfactant is reduced, it was recognized that sodium surfactin tends to be suitable for emulsification by an amino-modified silicone oil.

### Example 3

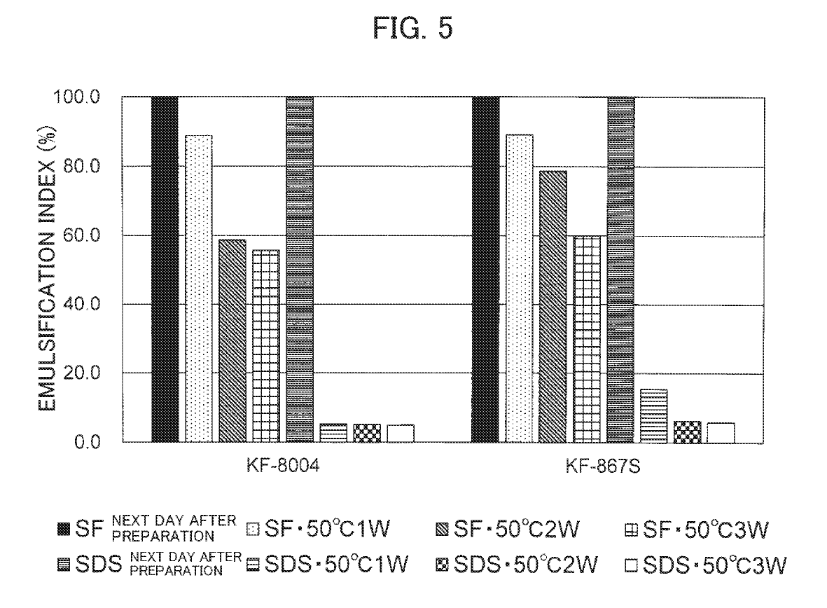

An experiment was carried out in the same manner as Example 1 above except that the amino-modified silicone oil shown in Table 2 was used. The result is shown in FIG. 5.

TABLE 2

| | Product | Viscosity |
|---|---|---|
| Component name | name | (mm²/s) |
| Aminoethylaminopropylmethylsiloxane | KF-8004 | 800 |
| dimethylsiloxane copolymer | | |
| Aminoethylaminopropylmethylsiloxane | KF-867S | 1300 |
| dimethylsiloxane copolymer | | |

Like the result shown in FIG. 5, even in the case of a separate amino-modified silicone oil (product names of “KF-8004” and “KF-867S”), it was shown that the emulsifying action is exhibited by sodium dodecyl sulfate immediately after mixing, but the emulsified state was not maintained. On the other hand, it was clear that the emulsified state is relatively stably maintained by sodium surfactin.

### Example 4

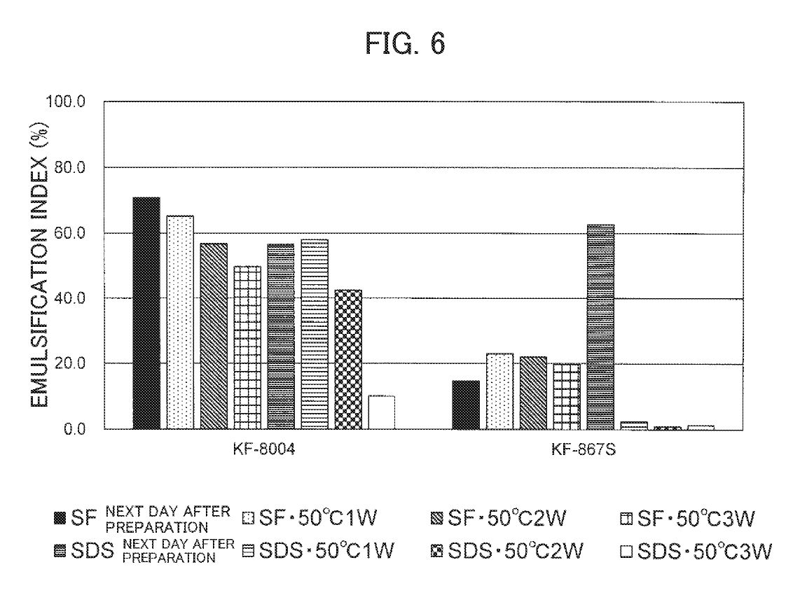

Furthermore, an experiment was carried out in the same manner as Example 3 above except that the concentration of the surfactant is reduced to 0.5 mM with respect to the entire mixture. The result is shown in FIG. 6.

Like the result shown in FIG. 6, in the case of the amino-modified silicone oil with product name of “KF-867S”, the emulsified state cannot be also maintained by sodium dodecyl sulfate; on the other hand, although the emulsifying effect by sodium surfactin was reduced, the emulsified state was maintained. Furthermore, also in the case of the amino-modified silicone oil with product name of “KF-8004”, the emulsified state was relatively maintained by sodium surfactin.

According to the experiments in the above, it was proved that sodium surfactin not only can suitably emulsify the mixture of an aqueous solvent and an amino-modified silicone oil but also can stably maintain the emulsified state.

Although the disclosure has been described with respect to only a limited number of embodiments, those skilled is the art, having benefit of this disclosure, will appreciate that various other embodiments may be devised without departing from the scope of the present invention. Accordingly, the scope of the invention should be limited only by the attached claims.

What is claimed is:

1. An emulsified composition, comprising:
as amino-modified silicone oil;
an aqueous solvent; and
a surfactin,
wherein the surfactin is represented by the following formula: 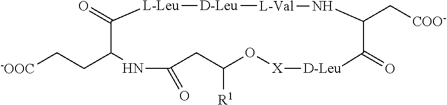
where X is an amino acid residue selected from the group consisting of a leucine residue, an isoleucine residue, and a valine residue; and R¹ is a C₉₋₁₈ alkyl group.

2. The composition according to claim 1, further comprising an oily component.

3. The composition according to claim 1, wherein the surfactin is contained in a concentration of 0.1 mM or more and 50 mM or less.

4. A cosmetic comprising the emulsified composition according to claim 1.
